(19) United States
(12) Patent Application Publication
(10) Pub. No.: US 2008/0114020 A1
(43) Pub. Date: May 15, 2008

(54) Difluoro-Substituted Imidazopyridines

(75) Inventors: Wilm Buhr, konstanz (DE); Peter Jan Zimmermann, Radolfzell (DE); Christof Brehm, Konstanz (DE); Adreas Palmer, Singen (DE); M.Vittoria Chiesa, Konstanz (DE); Wolfgang-Alexander Simon, Konstanz (DE); Stefan Postius, Konstanz (DE); Wolfgang Kromer, Konstanz (DE); Georg Rast, Singen (DE); Udo Doelling, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 11/663,925

(22) PCT Filed: Oct. 13, 2005
(86) PCT No.: PCT/EP05/55223

§ 371 Date: Apr. 26, 2007

(30) Foreign Application Priority Data
Oct. 15, 2004 (EP) 04105095.6

(51) Int. Cl. A61K 31/437; C07D 491/147; A61P 1/04

(52) U.S. Cl. 514/293; 546/83

(57) ABSTRACT

The invention provides compounds of the formula (I)

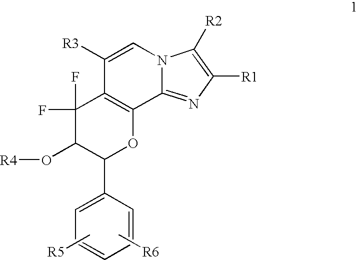

in which the substituents and symbols are as defined in the description. The compounds inhibit the secretion of gastric acid.

### TECHNICAL FIELD

The invention relates to novel compounds which are used in the pharmaceutical industry as active compounds for the production of medicaments.

### BACKGROUND ART

U.S. Pat. No. 4,468,400 describes tricyclic imidazo[1,2-a]pyridines having different ring systems fused to the imidazopyridine skeleton, which compounds are said to be suitable for treating peptide ulcer disorders. The International Patent Applications WO 95/27714, WO 98/42707, WO 98/54188, WO 00/17200, WO 00/26217, WO 00/50037, WO 00/63211, WO 01/72756, WO 01/72754, WO 01/72755, WO 01/72757, WO 02/34749, WO 03/014120, WO 03/016310, WO 03/014123, WO 03/068774, WO 03/091253 and 05/058325, disclose tricyclic imidazopyridine derivatives having a very specific substitution pattern, which compounds are likewise said to be suitable for treating gastrointestinal disorders.

The International Patent Application WO 04/099203 describes precursors for the tricyclic imidazopyridines disclosed in the patent applications mentioned above.

### DISCLOSURE OF INVENTION

### Technical Problem

A whole series of compounds are known from the prior art which inhibit gastric acid secretion by blockade of the H+/K+-ATPase. The compounds designated as proton pump inhibitors (PPI's), for example omeprazole, esomeprazole, lansoprazole, pantoprazole or rabeprazole, bind irreversibly to the H+/K+-ATPase. PPI's are available as therapeutics for a long time already. A new class of compounds designated as reversible proton pump inhibitors (rPPI's) or as acid pump antagonists (APA's) bind reversibly to the H+/K+-ATPase. Although rPPI's or APA's are known for more than 20 years and many companies are engaged in their development, no rPPI or APA is at present available for therapy. The technical problem underlying the present invention is therefore to provide acid pump antagonists which can be used in therapy.

### Technical Solution

The invention relates to compounds of the formula 1

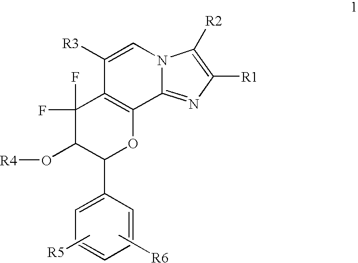

in which

R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkinyl, fluoro-1-4C-alkyl, fluoro-1-4C-alkoxy-1-4C-alkyl or hydroxy-1-4C-alkyl.

R2 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 2-4C-alkenyl, 2-4C-alkinyl, fluoro-1-4C-alkyl, fluoro-1-4C-alkoxy-1-4C-alkyl or hydroxy-1-4C-alkyl,

R3 is hydrogen, halogen, 1-4C-alkyl, fluoro-1-4C-alkyl, 3-7C-cycloalkyl, 3-7C-cycloalkyl-1-4C-alkyl, 1-4C-alkoxy-1-4C-alkyl, fluoro-1-4C-alkoxy-1-4C-alkyl or the group -CO-NR31 R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,

R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,

R5 is hydrogen, halogen, 1-4C-alkyl or fluoro-1-4C-alkyl

R6 is hydrogen, halogen, 1-4C-alkyl or fluoro-1-4C-alkyl

and the salts of these compounds.

Halogen within the meaning of the invention is bromo, chloro and fluoro. 1-4C-Alkyl represents straight-chain or branched alkyl groups having 1 to 4 carbon atoms. Examples which may be mentioned are the butyl, isobutyl, sec-butyl, tert-butyl, propyl, isopropyl, ethyl and the methyl group.

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkyl-1-4C-alkyl represents one of the aforementioned 1-4C-alkyl groups, which is substituted by one of the aforementioned 3-7C-cycloalkyl groups. Examples which may be mentioned are the cyclopropylmethyl, the cyclohexylmethyl and the cyclohexylethyl group.

1-4C-Alkoxy represents groups, which in addition to the oxygen atom contain a straight-chain or branched alkyl group having 1 to 4 carbon atoms. Examples which may be mentioned are the butoxy, isobutoxy, sec-butoxy, tert-butoxy, propoxy, isopropoxy and preferably the ethoxy and methoxy group.

1-4C-Alkoxy-1-4C-alkyl represents one of the aforementioned 1-4C-alkyl groups, which is substituted by one of the aforementioned 1-4C-alkoxy groups. Examples which may be mentioned are the methoxymethyl, the methoxyethyl group and the butoxyethyl group.

1-4C-Alkoxycarbonyl (—CO-1-4C-alkoxy) represents a carbonyl group, to which one of the aforementioned 1-4C-alkoxy groups is bonded. Examples which may be mentioned are the methoxycarbonyl (CH3O—C(O)—) and the ethoxycarbonyl group (CH3CH2O—C(O)—).

2-4C-Alkenyl represents straight-chain or branched alkenyl groups having 2 to 4 carbon atoms. Examples which may be mentioned are the 2-butenyl, 3-butenyl, 1-propenyl and the 2-propenyl group (allyl group).

2-4C-Alkinyl represents straight-chain or branched alkinyl groups having 2 to 4 carbon atoms. Examples which may be mentioned are the 2-butinyl, 3-butinyl, and preferably the 2-propinyl, group (propargyl group).

Fluoro-1-4C-alkyl represents one of the aforementioned 1-4C-alkyl groups, which is substituted by one or more fluorine atoms. Examples which may be mentioned are the trifluoromethyl group, the difluoromethyl, the 2-fluoroethyl, the 2,2-difluoroethyl or the 2,2,2-trifluoroethyl group.

Fluoro-1-4C-alkoxy-1-4C-alkyl represents one of the aforementioned 1-4C-alkyl groups, which is substituted by a fluoro-1-4C-alkoxy group. Fluoro-1-4C-alkoxy in this case represents one of the aforementioned 1-4C-alkoxy groups, which substituted by one or more fluorine atoms. Examples of fluoro-substituted 1-4C-alkoxy groups which may be mentioned are the 2-fluoro-ethoxy, 1,1,1,3,3,3-hexafluoro-2-propoxy, the 2-trifluoromethyl-2-propoxy, the 1,1,1-trifluoro-2-propoxy, the perfluoro-tertbutoxy, the 2,2,3,3,4,4,4-heptafluoro-1-butoxy, the 4,4,4-trifluoro-1-butoxy, the 2,2,3,3,3-penta-fluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy group. Examples of fluoro-1-4C-alkoxy-1-4C-alkyl radicals which may be mentioned are, 1,1,2,2-tetrafluoroethoxymethyl, the 2,2,2-trifluoroethoxymethyl, the trifluoromethoxymethyl, 2-fluoroethoxyethyl, the 1,1,2,2-tetrafluoroethoxyethyl, the 2,2,2-trifluoroethoxyethyl, the trifluoromethoxyethyl and preferably the difluoromethoxymethyl and the difluoromethoxyethyl radicals.

Hydroxy-1-4C-alkyl represents one of the aforementioned 1-4C-alkyl groups, which is substituted by a hydroxy group. Examples which may be mentioned are the hydroxymethyl, the 2-hydroxyethyl and the 3-hydroxypropyl group. Hydroxy-1-4C-alkyl within the scope of the invention is understood to include 1-4C-alkyl groups with two or more hydroxy groups. Examples which may be mentioned are the 3,4-dihydroxybutyl and in particular the 2,3-dihydroxypropyl group.

1-4C-Alkylcarbonyl represents a group, which in addition to the carbonyl group contains one of the aforementioned 1-4C-alkyl groups. An example which may be mentioned is the acetyl group.

1-4C-alkoxy-1-4C-alkylcarbonyl represents a group, which in addition to the carbonyl group contains one of the aforementioned 1-4C-alkoxy-1-4C-alkyl groups. Examples which may be mentioned are the methoxymethylcarbonyl (CH3-O—CH2-C(O)—), the ethoxymethylcarbonyl (CH3CH2-O—CH2-C(O)—) and the isobutoxymethylcarbonyl ((CH3)2CH—CH2-O—CH2-C(O)—) group.

Mono- or di-1-4C-alkylamino radicals contain, in addition to the nitrogen atom, one or two of the abovementioned 1-4C-alkyl radicals. Preference is given to di-1-4C-alkylamino and in particular to dimethyl-, diethyl- or diisopropylamino.

Mono- or di-1-4C-alkylamino-1-4C-alkylcarbonyl represents a 1-4C-alkylcarbonyl group, which is substituted by a mono- or di-1-4C-alkylamino group. Examples, which may be mentioned, are the dimethylamino-methyl-carbonyl ((CH3)2N—CH2-C(O)—) and the diethylamino-methylcarbonyl ((CH3CH2)2N—CH2-C(O)—) group or the methylamino-methyl-carbonyl (CH3N(H)—CH2-C(O)—) group.

Suitable salts of compounds of the formula 1 are—depending on the substitution—in particular all acid addition salts. Particular mention may be made of the pharmacologically acceptable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are employed in the salt preparation in an equimolar ratio or in a ratio differing therefrom, depending on whether the acid is a mono- or polybasic acid and on which salt is desired.

Pharmacologically unacceptable salts, which can be initially obtained, for example, as process products in the preparation of the compounds according to the invention on an industrial scale, are converted into pharmacologically acceptable salts by processes known to the person skilled in the art.

It is known to the person skilled in the art that the compounds according to the invention and their salts can, for example when they are isolated in crystalline form, comprise varying amounts of solvents. The invention therefore also embraces all solvates and, in particular, all hydrates of the compounds of the formula 1, and all solvates and, in particular, all hydrates of the salts of the compounds of the formula 1.

The compounds of the formula 1 have at least two centers of chirality in the skeleton. The invention thus provides all feasible stereoisomers in any mixing ratio, including the pure stereoisomers, which are a preferred subject matter of the invention.

One embodiment (embodiment a) according to the invention to be mentioned comprises compounds wherein R3 is hydrogen and wherein R1, R2, R4, R5 and R6 have the meanings as indicated in the outset.

Another embodiment (embodiment b) according to the invention to be mentioned comprises compounds wherein R3 is 1-4C-alkoxy-1-4C-alkyl and wherein R1, R2, R4, R5 and R6 have the meanings as indicated in the outset.

Another embodiment (embodiment c) according to the invention to be mentioned comprises compounds wherein R5 and R6 are each hydrogen and wherein R1, R2, R3 and R4 have the meanings as indicated in the outset.

The invention also relates to compounds of the formula 1, in which

R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkinyl, fluoro-1-4C-alkyl, fluoro-1-4C-alkoxy-1-4C-alkyl or hydroxy-1-4C-alkyl.

R2 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 2-4C-alkenyl, 2-4C-alkinyl, fluoro-1-4C-alkyl, fluoro-1-4C-alkoxy-1-4C-alkyl or hydroxy-1-4C-alkyl,

R3 is hydrogen, halogen, 1-4C-alkyl, fluoro-1-4C-alkyl, 3-7C-cycloalkyl, 3-7C-cycloalkyl-1-4C-alkyl, 1-4C-alkoxy-1-4C-alkyl, fluoro-1-4C-alkoxy-1-4C-alkyl or the group —CO—NR31 R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,

R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,

R5 is hydrogen, halogen, 1-4C-alkyl or fluoro-1-4C-alkyl

R6 is hydrogen, halogen, 1-4C-alkyl or fluoro-1-4C-alkyl

and the salts of these compounds.

Compounds of the formula 1 which are to be mentioned are those compounds of the formula 1, where

R1 is hydrogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,

R2 is hydrogen, halogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,

R3 hydrogen, halogen, 1-4C-alkyl, fluoro-1-4C-alkyl, 3-7C-cycloalkyl, 1-4C-alkoxy-1-4C-alkyl, fluoro-1-4C-alkoxy-1-4C-alkyl or the group —CO—NR31 R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,

R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,

R5 is hydrogen, halogen, 1-4C-alkyl or fluoro-1-4C-alkyl

R6 is hydrogen, halogen, 1-4C-alkyl, fluoro-1-4C-alkyl

and the salts of these compounds.

Compounds of the formula 1 which are also to be mentioned are those compounds of the formula 1, where

R1 is hydrogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,

R2 is hydrogen, halogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,

R3 hydrogen, halogen, 1-4C-alkyl, fluoro-1-4C-alkyl, 3-7C-cycloalkyl, 1-4C-alkoxy-1-4C-alkyl, fluoro-1-4C-alkoxy-1-4C-alkyl or the group —CO—NR31R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,

R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,

R5 is hydrogen, halogen, 1-4C-alkyl or fluoro-1-4C-alkyl

R6 is hydrogen, halogen, 1-4C-alkyl, fluoro-1-4C-alkyl

and the salts of these compounds.

Compounds of the formula 1 which are to be particularly mentioned are those compounds of the formula 1, where

R1 is 1-4C-alkyl,

R2 is hydrogen or 1-4C-alkyl,

R3 is hydrogen or 1-4C-alkoxy-1-4C-alkyl,

R4 is hydrogen, 1-4C-alkyl, 3-7C-cycloalkyl, 1-4C-alkoxy-1-4C-alkyl, fluoro-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,

R5 is hydrogen,

R6 is hydrogen

and the salts of these compounds.

Compounds of the formula 1 which are also to be particularly mentioned are those compounds of the formula 1, where

R1 is 1-4C-alkyl,

R2 is hydrogen or 1-4C-alkyl,

R3 is hydrogen or 1-4C-alkoxy-1-4C-alkyl,

R4 is hydrogen, 1-4C-alkyl, 3-7C-cycloalkyl, 1-4C-alkoxy-1-4C-alkyl, fluoro-1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,

R5 is hydrogen,

R6 is hydrogen

and the salts of these compounds.

Compounds of the formula 1 which are to be emphasized are those compounds of the formula 1, where

R1 is 1-4C-alkyl,

R2 is hydrogen or 1-4C-alkyl,

R3 is hydrogen,

R4 is hydrogen, 1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,

R5 is hydrogen,

R6 is hydrogen

and the salts of these compounds.

Compounds of the formula 1 which are also to be emphasized, are those compounds of the formula 1, where

R1 is 1-4C-alkyl,

R2 is 1-4C-alkyl,

R3 is hydrogen,

R4 is hydrogen, 1-4C-alkyl, 1-4C-alkoxy-1-4C-alkyl, hydroxy-1-4C-alkyl or 1-4C-alkylcarbonyl,

R5 is hydrogen,

R6 is hydrogen

and the salts of these compounds.

Among the compounds of the formula 1 according to the invention, emphasis is given to the optically pure compounds of the formula 1a

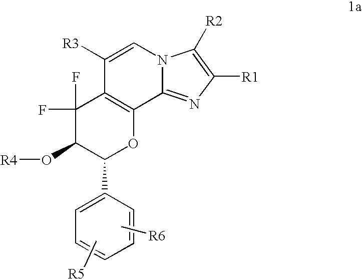

in which

R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkinyl, fluoro-1-4C-alkyl, fluoro-1-4C-alkoxy-1-4C-alkyl or hydroxy-1-4C-alkyl.

R2 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 2-4C-alkenyl, 2-4C-alkinyl, fluoro-1-4C-alkyl, fluoro-1-4C-alkoxy-1-4C-alkyl or hydroxy-1-4C-alkyl,

R3 is hydrogen, halogen, 1-4C-alkyl, fluoro-1-4C-alkyl, 3-7C-cycloalkyl, 3-7C-cycloalkyl-1-4C-alkyl, 1-4C-alkoxy-1-4C-alkyl, fluoro-1-4C-alkoxy-1-4C-alkyl or the group -CO-NR31 R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,

R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,

R5 is hydrogen, halogen, 1-4C-alkyl or fluoro-1-4C-alkyl

R6 is hydrogen, halogen, 1-4C-alkyl or fluoro-1-4C-alkyl

and the salts of these compounds.

Among the compounds of the formula 1 according to the invention, emphasis is also given to the optically pure compounds of the formula 1a in which

R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkinyl, fluoro-1-4C-alkyl, fluoro-1-4C-alkoxy-1-4C-alkyl or hydroxy-1-4C-alkyl.

R2 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 2-4C-alkenyl, 2-4C-alkinyl, fluoro-1-4C-alkyl, fluoro-1-4C-alkoxy-1-4C-alkyl or hydroxy-1-4C-alkyl,

R3 is hydrogen, halogen, 1-4C-alkyl, fluoro-1-4C-alkyl, 3-7C-cycloalkyl, 3-7C-cycloalkyl-1-4C-alkyl, 1-4C-alkoxy-1-4C-alkyl, fluoro-1-4C-alkoxy-1-4C-alkyl or the group -CO-NR31 R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,

R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,

R5 is hydrogen, halogen, 1-4C-alkyl or fluoro-1-4C-alkyl

R6 is hydrogen, halogen, 1-4C-alkyl or fluoro-1-4C-alkyl and the salts of these compounds.

Compounds of the formula 1 a which are to be mentioned are those, where

R1 is hydrogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,

R2 is hydrogen, halogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,

R3 hydrogen, halogen, 1-4C-alkyl, fluoro-1-4C-alkyl, 3-7C-cycloalkyl, 1-4C-alkoxy-1-4C-alkyl, fluoro-1-4C-alkoxy-1-4C-alkyl or the group —CO—NR31R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,

R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,

R5 is hydrogen, halogen, 1-4C-alkyl or fluoro-1-4C-alkyl

R6 is hydrogen, halogen, 1-4C-alkyl, fluoro-1-4C-alkyl

and the salts of these compounds.

Compounds of the formula 1a which are also to be mentioned are those, where

R1 is hydrogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,

R2 is hydrogen, halogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,

R3 hydrogen, halogen, 1-4C-alkyl, fluoro-1-4C-alkyl, 3-7C-cycloalkyl, 1-4C-alkoxy-1-4C-alkyl, fluoro-1-4C-alkoxy-1-4C-alkyl or the group -CO-NR31 R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,

R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,

R5 is hydrogen, halogen, 1-4C-alkyl or fluoro-1-4C-alkyl

R6 is hydrogen, halogen, 1-4C-alkyl, fluoro-1-4C-alkyl

and the salts of these compounds.

Compounds of the formula 1a which are to be particularly mentioned are those, where

R1 is 1-4C-alkyl,

R2 is hydrogen or 1-4C-alkyl,

R3 is hydrogen or 1-4C-alkoxy-1-4C-alkyl,

R4 is hydrogen, 1-4C-alkyl, 3-7C-cycloalkyl, 1-4C-alkoxy-1-4C-alkyl, fluoro-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,

R5 is hydrogen,

R6 is hydrogen

and the salts of these compounds.

Compounds of the formula 1a which are also to be particularly mentioned are those, where

R1 is 1-4C-alkyl,

R2 is hydrogen or 1-4C-alkyl,

R3 is hydrogen or 1-4C-alkoxy-1-4C-alkyl,

R4 is hydrogen, 1-4C-alkyl, 3-7C-cycloalkyl, 1-4C-alkoxy-1-4C-alkyl, fluoro-1-4C-alkyl, hydroxy-1-4C-alkyl, 1-4C-alkoxy-1-4C-alkylcarbonyl, mono- or di-1-4C-alkylamino-1-4C-alkylcarbonyl or 1-4C-alkylcarbonyl,

R5 is hydrogen,

R6 is hydrogen

and the salts of these compounds.

Compounds of the formula 1a which are to be emphasized, are those compounds, where

R1 is 1-4C-alkyl,

R2 is hydrogen or 1-4C-alkyl,

R3 is hydrogen

R4 is hydrogen, 1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,

R5 is hydrogen,

R6 is hydrogen

and the salts of these compounds.

Compounds of the formula 1a which are also to be emphasized, are those compounds, where

R1 is 1-4C-alkyl,

R2 is 1-4C-alkyl,

R3 is hydrogen,

R4 is hydrogen, 1-4C-alkyl, 1-4C-alkoxy-1-4C-alkyl, hydroxy-1-4C-alkyl or 1-4C-alkylcarbonyl,

R5 is hydrogen,

R6 is hydrogen

and the salts of these compounds.

Particular emphasis is given to the compounds of the formula 1 given as final products in the examples, and the salts of these compounds.

The compounds according to the invention can be synthesized from corresponding starting compounds, for example according to the reaction schemes given below. The synthesis is carried out in a manner known to the expert, for example as described in more detail in the following examples.

Compounds of the formula 1 can be obtained, for example, by fluorination of compounds of formula 2 and further derivatization reactions (see scheme 1). The fluorination can be performed with deoxo-fluorination reagents like for example with reagents like DAST (dialkylaminosulfur trifluoride), Deoxo-Fluor (Bis(2-methoxyethyl)aminosulfur trifluoride) or other standard deoxo-fluorination reagents under standard condition.

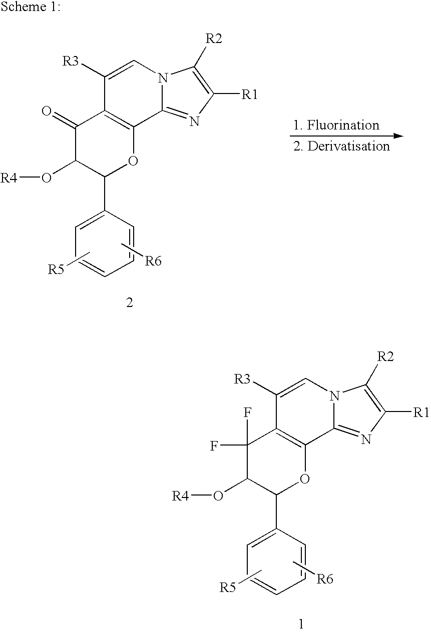

Compounds of the formula 1 initially obtained after fluorination can be transformed to further compounds of the formula 1 by methods known to the expert. Examples for these further derivatizations are transformation of group R4 into another group R4, like for example the etherification of compounds of the formula 1 with R4=OH or cleavage of an ester in a compound of the formula 1 with R4 =1-4C-alkylcarbonyl and further derivatizations known to a person skilled in the art.

The synthesis as outlined in scheme 2 leads to the preferred optically pure compound of the formula 1a.

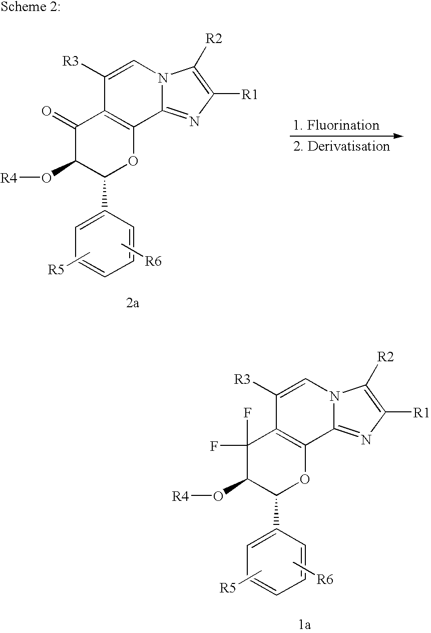

Compounds of the formula 2 and 2a are known, for example from WO 98/54188, WO 01/72755, WO 02/34749 or WO 04/099203 or can be prepared in a similar manner by methods known to the expert.

The compounds of the formula 2 and 2a with R4=1-4C-alkylcarbonyl can be prepared according to the following reaction scheme 3 as shown in an exemplary manner for compounds of the formula 2a. This procedure is disclosed in more detail in WO 04/099203:

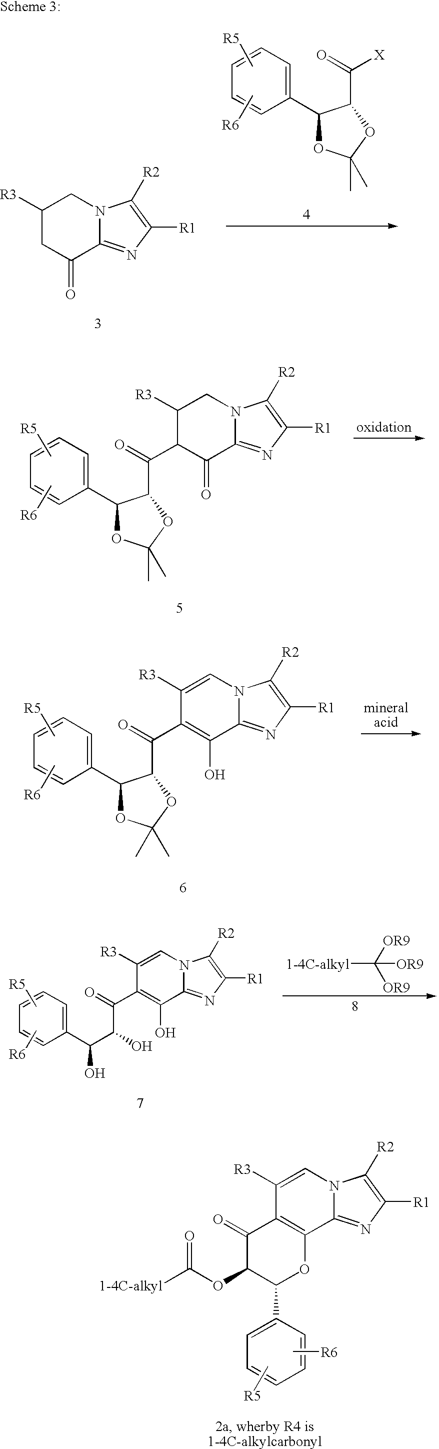

Compounds of the formula 6 are known (see for example WO 01/72755, WO 02/34749 or WO 04/099203) or they can be prepared in a known manner in analogy to known compounds from known starting materials, for example as shown in scheme 3 above. The group X in compounds of the formula 4 is a suitable leaving group, like for example halogen, preferably chlorine, or a 1-4C-alkoxy group, preferably methoxy. Treatment of compounds of the formula 6 with a mineral acid, like for example phosphoric acid, hydrochloric acid or sulphuric acid leads to compounds of the formula 7, which can be converted to compounds of the formula 2a by use of an ortho-ester of the formula 8, wherein R9 is for example a 1-4C-alkyl group. This cyclization is performed in manner known to the expert, for example in analogy to the process described in Drugs Fut. 2001, 26(6), 590 or WO 04/099203.

### Advantageous Effects

The excellent gastric protective action and the gastric acid secretion-inhibiting action of the compounds according to the invention can be demonstrated in investigations on animal experimental models. The compounds of the formula 1 according to the invention investigated in the model mentioned below have been provided with numbers which correspond to the numbers of these compounds in the examples.

### Testing of the Secretion-Inhibiting Action on the Perfused Rat Stomach

In Table A which follows, the influence of the compounds of the formula 1 according to the invention on the pentagastrin-stimulated acid secretion of the perfused rat stomach after intraduodenal administration in vivo is shown.

TABLE A

| | Dose | Inhibition of |
|---|---|---|
| | (μmol/kg) | acid secretion |
| No. | i.d. | (%) |
| 1 | 1 | >85 |
| 2 | 1 | >85 |
| 8 | 1 | >85 |
| 9 | 1 | >85 |
| 10 | 1 | >85 |

### Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01 N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion. The body temperature of the animals was kept at a constant 37.8-38° C. by infrared irradiation and heat pads (automatic, stepless control by means of a rectal temperature sensor).

### MODE(S) FOR CARRYING OUT THE INVENTION

The examples below serve to illustrate the invention in more detail without limiting it. Further compounds of the formula 1 whose preparation is not described explicitly can likewise be prepared in an analogous manner or in a manner known per se to the person skilled in the art, using customary process techniques. The compounds named expressly as examples, and the salts of these compounds, are preferred subject matter of the invention. The abbreviation min stands for minute(s), h stands for hour(s), m.p. stands for melting point and ee for enantiomeric excess.

### I. Final Products of Formula 1

### 1. (8R,9R)-7,7-Difluoro-2,3-dimethyl-8-hydroxy-9-phenyl-7H-8,9-dihydro-pyrano[2,3-c]imidazo[1,2-a]pyridine

To a at −10° C. cooled suspension of 8.15 g (26.4 mmol) (8R,9R)-2,3-dimethyl-8-hydroxy-9-phenyl-7H-8,9-dihydro-pyrano[2,3-c]imidazo[1,2-a]pyridin-7-one were added dropwise 10.65 g (66.0 mmol) DAST (diethylaminosulfur trifluoride) and it was stirred for further 30 min at −10° C. and for 2 h at 25° C. Subsequently the reaction was quenched by pouring out into ice water and the reaction mixture was basified by adding sodium hydroxide solution (6 N) and saturated sodium hydrogen carbonate solution. The mixture was extracted with dichloromethane / methanol (13/1) two times. The combined organic layers were concentrated in vacuo, the crude product was purified by column chromatography (dichloromethane/methanol: 100/1 to 100/3) and reslurried in dichloromethane to give 4.16 g (12.6 mmol/48%) of the title product with a melting point of 255° C. (dichloromethane).

### 2. (8R,9R)-8-Acetoxy-7,7-difluoro-2,3-dimethyl-9-phenyl-7H-8,9-dihydro-pyrano[2,3-c]imidazo[1,2-a]pyridine

To a at −10° C. cooled solution of 5.00 g (31.0 mmol) DAST (diethylaminosulfur trifluoride) was added in portions 1.00 g (2.80 mmol) (8R,9R)-8-acetoxy-2,3-dimethyl-9-phenyl-7H-8,9-dihydro-pyrano[2,3-c]imidazo[1,2-a]pyridin-7-one and it was stirred for further 30 min at −10° C. and for 4 d at 25° C. Subsequently the reaction was quenched by pouring out dropwise into an ice cooled saturated sodium hydrogen carbonate solution. The mixture was extracted with dichloromethane three times. The combined organic layers were concentrated in vacuo and the crude product was purified by column chromatography three times (triethylamine/diethyl ether/diisopropyl ether). The product was reslurried in diethyl ether to give 0.24 g (0.64 mmol/23%) of the title product with a melting point of 200° C. (diethyl ether).

### 3. (8R,9R)-7,7-Difluoro-2,3-dimethyl-8-(2-methoxyethoxy)-9-phenyl-7H-8,9-dihydro-pyrano[2,3-c]imidazo[1,2-a]pyridine

To a stirred suspension of 1.40 g (4.24 mmol) (8R,9R)-7,7-difluoro-2,3-dimethyl-8-hydroxy-9-phenyl-7H-8,9-dihydro-pyrano[2,3-c]imidazo[1,2-a]pyridine in THF (45 ml) were added in portions 0.19 g (4.66 mmol) sodium hydride and it was stirred for further 0.5 h. Afterwards 0.97 g (4.66 mmol) 2-methoxy-ethyl trifluoromethanesulphonate was added and the reaction mixture was stirred for further 1 h. Subsequently the mixture was poured out into saturated ammonium chloride solution and was extracted with ethyl acetate three times. The combined organic layers were concentrated in vacuo and the crude product was purified by column chromatography (dichloromethane/methanol: 13/1). This product was crystallized from diethyl ether to give 0.47 g (1.21 mmol / 29%) of the title product with a melting point of 109° C. (diethyl ether).

### 4. (8R,9R)-7,7-Difluoro-2,3-dimethyl-8-(2-hydroxyethoxy)-9-phenyl-7H-8,9-dihydro-pyrano[2,3-c]imidazo[1,2-a]pyridine

To a solution of 0.90 g (2.30 mmol) (8R,9R)-7,7-difluoro-2,3-dimethyl-8-(2-methoxyethoxy)-9-phenyl-7H-8,9-dihydro-pyrano[2,3-c]imidazo[1,2-a]pyridine in dichloromethane (2.0 ml) were added 46.0 ml of a 15-crown-5 (1,4,7,10,13-pentaoxacyclopentadecane) solution (0.3 M in dichloromethane saturated with sodium iodide ). The reaction mixture was cooled to −40° C. and 7.00 ml (7.00 mmol) boron tribromide (1 M in dichloromethane) were added and it was stirred for further 2.5 h at −30° C. Subsequently the mixture was poured out into saturated sodium hydrogen carbonate solution and was extracted with dichloromethane three times. The combined organic layers were washed with water and concentrated in vacuo. The crude product was purified by column chromatography (dichloromethane/methanol: 13/1) and diethyl ether to give 0.56 g (1.50 mmol / 65%) of the title product with a melting point of 146° C. (diethyl ether).

### 5. (8R,9R)-7,7-Difluoro-2,3-dimethyl-8-ethoxy-9-phenyl-7H-8,9-dihydro-pyrano[2,3-c]imidazo[1,2-a]pyridine

To a stirred suspension of 1.00 g (3.03 mmol) (8R,9R)-7,7-difluoro-2,3-dimethyl-8-hydroxy-9-phenyl-7H-8,9-dihydro-pyrano[2,3-c]imidazo[1,2-a]pyridine in THF (30 ml) was added in portions 0.13 g (3.33 mmol) sodium hydride and it was stirred for further 0.5 h. Afterwards 0.59 g (3.33 mmol) ethoxy trifluoromethanesulphonate was added and the reaction mixture was stirred for further 1 h. Subsequently the mixture was poured out into saturated ammonium chloride solution, the organic layer was separated and the water layer was extracted with ethyl acetate three times. The combined organic layers were concentrated in vacuo and the crude product was purified by column chromatography (dichloromethane/methanol: 100/3). This product was crystallized from diisopropyl ether to give 0.51 g (1.42 mmol /46%) of the title product with a melting point of 147° C. (diisopropyl ether).

### 6. (8R,9R)-7,7-Difluoro-2,3-dimethyl-8-methoxy-9-phenyl-7H-8,9-dihydro-pyrano[2,3-c]imidazo[1,2-a]pyridine

To a stirred suspension of 1.40 g (4.24 mmol) (8R,9R)-7,7-difluoro-2,3-dimethyl-8-hydroxy-9-phenyl-7H-8,9-dihydro-pyrano[2,3-c]imidazo[1,2-a]pyridine in THF (30 ml) was added in portions 0.13 g (3.33 mmol) sodium hydride and it was stirred for further 0.5 h. Afterwards 0.55 g (3.33 mmol) methoxy trifluoromethanesulphonate was added and the reaction mixture was stirred for further 1 h. Subsequently the mixture was poured out into saturated ammonium chloride solution, the organic layer was separated and the water layer was extracted with ethyl acetate three times. The combined organic layers were concentrated in vacuo and the crude product was purified by column chromatography three times (dichloromethane/methanol: 100/1). This product was crystallized from diisopropyl ether to give 0.51 g (1.48 mmol/49%) of the title product with a melting point of 157° C. (diisopropyl ether).

### 7. (8R,9R)-7,7-Difluoro-8-hydroxy-2-methyl-9-phenyl-7H-8,9-dihydro-pyrano[2,3-c]imidazo[1,2-a]pyridine

To a suspension of 1.00 g (3.40 mmol) (8R,9R)-8-hydroxy-2-methyl-9-phenyl-7H-8,9-dihydro-pyrano[2,3-c]imidazo[1,2-a]pyridin-7-one is added dropwise 5.00 ml (18.8 mmol) BAST [bis(2-methoxyethyl)amino]sulfur trifluoride) and it is stirred for further 5 h at 25° C. Subsequently the reaction is quenched by pouring out into a saturated sodium hydrogen carbonate solution. The organic layer is separated and the water layer is extracted with dichloromethane methanol 100/3 three times. The combined organic layers are concentrated in vacuo and the crude product is purified by column chromatography (dichloromethane/methanol: 100/1 to 100/3) and reslurried in dichloromethane to give 0.27 g (0.85 mmol/25%) of the title product with a melting point of 226° C. (dichloromethane).

### 8. (8R,9R)-7,7-Difluoro-2,3-dimethyl-8-(2-methoxy-acetyloxy)-9-phenyl-7H-8,9-dihydro-pyrano[2,3-c]imidazo[1,2-a]pyridine

To a suspension of 1.00 g (3.03 mmol) (8R,9R)-7,7-difluoro-2,3-dimethyl-8-hydroxy-9-phenyl-7H-8,9-dihydro-pyrano[2,3-c]imidazo[1,2-a]pyridine in dichloromethane (10 ml) is added 2.10 ml (15.0 mmol) triethylamine, 50.0 mg (0.40 mmol) 4-dimethylaminopyridine and 1.02 g (9.08 mmol) methoxyacetyl chloride diluted in dichloromethane (2 ml). The reaction is stirred for further 1 h at 25° C. Subsequently the mixture is poured out into water and extracted with dichloromethane three times. The combined organic layers are concentrated in vacuo and purified by column chromatography (dichloromethane/methanol: 100/3). The product is crystallized from diethyl ether to give 0.95 g (2.36 mmol/78%) of the title product as a colourless solid with a melting point of 167° C. (diethyl ether).

### 9. (8R,9R)-7,7-Difluoro-2,3-dimethyl-8-(2-dimethylamino-acetyloxy)-9-phenyl-7H-8,9-dihydro-pyrano[2,3-c]imidazo[1,2-a]pyridine

A suspension of 0.64 g (6.06 mmol) N,N-dimethylglycine and 1.01 g (6.06 mmol) 1.1′-carbodiimidazole in THF (20 ml) is stirred at 45° C. for 2 h. Afterwards 1.00 g (3.03 mmol) (8R,9R)-7,7-difluoro-2,3-dimethyl-8-hydroxy-9-phenyl-7H-8,9-dihydro-pyrano[2,3-c]imidazo[1,2-a]pyridine is added portionwise at 25° C. and the reaction mixture is stirred for further 4 h. Subsequently the mixture is poured out into water and extracted with ethyl acetate three times. The combined organic layers are concentrated in vacuo and purified by column chromatography (ethyl acetate: 100). The product is reslurried from diethyl ether to give 0.85g (2.05 mmol/62%) of the title product as a colourless solid with a melting point of 163° C. (diethyl ether).

### 10. (8R,9R)-7,7-Difluoro-2,3-dimethyl-8-(methoxy-carbonyloxy)-9-phenyl-7H-8,9-dihydro-pyrano[2,3-c]imidazo[1,2-a]pyridine

To a at −10° C. cooled suspension of 1.00 g (3.03 mmol) (8R,9R)-7,7-difluoro-2,3-dimethyl-8-hydroxy-9-phenyl-7H-8,9-dihydro-pyrano[2,3-c]imidazo[1,2-a]pyridine in dichloromethane (20 ml) is added 2.10 ml (15.0 mmol) triethylamine, 50.0 mg (0.40 mmol) 4-dimethylaminopyridine and 0.71 ml (9.08 mmol) methylchloroformate diluted in dichloromethane (2 ml). The reaction is stirred for further 3 h at 250C. Subsequently the mixture is poured out into a saturated ammonium chloride solution and is extracted with dichloromethane three times. The combined organic layers are concentrated in vacuo and purified by column chromatography (dichloromethane/methanol: 100/1). The product is crystallized from acetone to give 0.70 g (1.80 mmol/60%) of the title product as a colourless solid with a melting point of 222° C. (acetone).

### II. Starting Materials

### A. 2,3-Dimethyl-7-[(2R,3S)-2,3-diyhdroxy-3-phenylpropan-1-on-1-yl]imidazo[1,2-a]-pyridin-8-ol

10 g (0.027 mol) of 2,3-Dimethyl-7-[(2R,3S)-2,3-O,O -isopropyliden-2,3-dioxy-3-phenylpropan-1-on-1-yl]imidazo[1,2-a]-pyridin-8-ol were dissolved in 50 ml of 6 molar hydrochloric acid. After stirring for 30 min at room temperature, the reaction mixture was cooled with an ice bath and neutralized to pH 6.5 using a 6 molar sodium hydroxide solution. The resulting residue was filtered off, washed with water and dried in vacuum at 55° C. The crude product was purified by chromatography on silica gel (eluent dichloromethane/methanol: 5/1) to give 5.77 g (0.018 mol, yield 65%) of the title compound as ochre crystals.

### ¹H-NMR (CDCl₃): (ppm)=2.25 (3H, s), 2.38 (3H, s), 5.32 (1H, d), 5.55 (1H, d), 7.13 (1H, d), 7.18-7.25 (3H, m), 7.51 (2H, dd), 7.70 (1 H, d).

### B. (8R,9R)-8-Acetoxy-2,3-dimethyl-9-phenyl-7H-8,9-dihydro-pyrano[2,3-c]imidazo[1,2- a]-pyridin-7-on

3.18 ml (0.025 mol) of orthoacetic acid trimethyl ester, 0.16 g (0.0006 mol) of pyridinium-p- toluenesulfonate and 0.28 ml (0.007 mol) of formic acid were added at room temperature to a solution of 2 g (0.006 mol) of 2,3-dimethyl-7-[(2R,3S)-2,3-diyhdroxy-3-phenylpropan-1-on-1-yl]imidazo-[1,2-a]-pyridin-8-ol in 40 ml of dichloromethane. After stirring for 16 h at room temperature, the reaction mixture was diluted with dichloromethane, washed with saturated sodium bicarbonate solution, dried over sodium sulphate and the solvent removed under vacuum. The crude product was purified by filtration over a thin layer of silica gel (solvent dichloromethane/methanol: 100/1). After removal of the solvent, 1.8 g (0.005 mol, yield 84%) of the title compound were obtained in form of colourless crystals. m. p.: 205-206° C. (diethyl ether / acetone).

### INDUSTRIAL APPLICABILITY

The compounds of the formula 1 and 1a and their salts (active compounds according to the invention) have valuable pharmacological properties which make them commercially utilizable. In particular, they exhibit marked inhibition of gastric acid secretion and an excellent gastric and intestinal protective action in warm-blooded animals, in particular humans. In this connection, the active compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, peptic ulcer, including peptic ulcer bleeding, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics, such as NSAIDs and COX-inhibitors), chemicals (e.g. ethanol), gastric acid or stress situations. “Gastric and intestinal protection” is understood to include, according to general knowledge, gastroesophageal reflux disease (GERD), the symptoms of which include, but are not limited to, heartburn and/or acid regurgitation.

In their excellent properties, the active compounds according to the invention surprisingly prove to be clearly superior to the compounds known from the prior art in various models in which the antiulcerogenic and the antisecretory properties are determined. On account of these properties, the active compounds according to the invention are outstandingly suitable for use in human and veterinary medicine, where they are used, in particular, for the treatment and/or prophylaxis of disorders of the stomach and/or intestine.

A further subject of the invention are therefore the active compounds according to the invention for use in the treatment and/or prophylaxis of the abovementioned diseases.

The invention likewise includes the use of the active compounds according to the invention for the production of medicaments which are employed for the treatment and/or prophylaxis of the abovementioned diseases.

The invention furthermore includes the use of the active compounds according to the invention for the treatment and/or prophylaxis of the abovementioned diseases.

A further subject of the invention are medicaments which comprise one or more compounds of the active compounds according to the invention.

The medicaments are prepared by processes which are known per se and familiar to the person skilled in the art. As medicaments, the pharmacologically active compounds according to the invention are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds according to the invention can be administered orally, parenterally or percutaneously.

In general, it has proven advantageous in human medicine to administer the active compound according to the invention in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the active compound according to the invention and/or their salts are to be used for the treatment of the abovementioned diseases, the pharmaceutical preparations can also contain one or more pharmacologically active constituents of other groups of medicaments, for example: tranquillizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino acids. To be emphasized in this connection is in particular the combination of the active compounds according to the invention with pharmaceuticals which inhibit acid secretion, such as, for example, H2 blockers (e.g. cimetidine, ranitidine), H+/K+ ATPase inhibitors (e.g. omeprazole, pantoprazole), or further with so-called peripheral anticholinergics (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, meziocillin, ampicillin, amoxicillin, cefalothin, cefoxitin, cefotaxime, imipenem, gentamycin, amikacin, erythromycin, ciprofloxacin, metronidazole, clarithromycin, azithromycin and combinations thereof (for example clarithromycin+metronidazole).

In view of their excellent gastric and intestinal protection action, the active compounds according to the invention are suited for a free or fixed combination with those medicaments (e.g. certain antiinflammatories and antirheumatics, such as NSAIDs), which are known to have a certain ulcerogenic potency. In addition, the active compounds according to the invention are suited for a free or fixed combination with motility-modifying drugs.

1. A compound of the formula 1 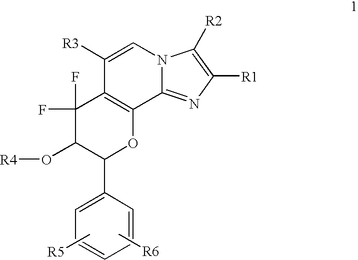
in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkinyl, fluoro-1-4C-alkyl, fluoro-1-4C-alkoxy-1-4C-alkyl or hydroxy-1-4C-alkyl,
R2 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 2-4C-alkenyl, 2-4C-alkinyl, fluoro-1-4C-alkyl, fluoro-1-4C-alkoxy-1-4C-alkyl or hydroxy-1-4C-alkyl,
R3 is hydrogen, halogen, 1-4C-alkyl, fluoro-1-4C-alkyl, 3-7C-cycloalkyl, 3-7C-cycloalkyl-1-4C-alkyl, 1-4C-alkoxy-1-4C-alkyl, fluoro-1-4C-alkoxy-1-4C-alkyl or the group —CO—NR31R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,
R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,
R5 is hydrogen, halogen, 1-4C-alkyl or fluoro-1-4C-alkyl,
R6 is hydrogen, halogen, 1-4C-alkyl or fluoro-1-4C-alkyl, or a salt thereof.

2. A compound of the formula 1 as claimed in claim 1, in which
R1 is hydrogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,
R2 is hydrogen, halogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,
R3 hydrogen, halogen, 1-4C-alkyl, fluoro-l-4C-alkyl,. 3-7C-cycloalkyl, 1-4C-alkoxy-1-4C-alkyl, fluoro-1-4C-alkoxy-1-4C-alkyl or the group —CO—NR31R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,
R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,
R5 is hydrogen, halogen, 1-4C-alkyl or fluoro-1-4C-alkyl,
R6 is hydrogen, halogen, 1-4C-alkyl or fluoro-1-4C-alkyl,
or a salt thereof

3. A compound of the formula 1 as claimed in claim 1, in which
R1 is 1-4C-alkyl,
R2 is hydrogen or 1-4C-alkyl,
R3 is hydrogen or 1-4C-alkoxy-1-4C-alkyl,
R4 is hydrogen, 1-4C-alkyl, 3-7C-cycloalkyl, 1-4C-alkoxy-1-4C-alkyl, fluoro-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,
R5 is hydrogen,
R6 is hydrogen,
or a salt thereof.

4. A compound of the formula 1, as claimed in claim 1, in which
R1 is 1-4C-alkyl,
R2 is hydrogen or 1-4C-alkyl,
R3 is hydrogen,
R4 is hydrogen, 1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,
R5 is hydrogen,
R6 is hydrogen,
or a salt thereof.

5. A compound of the formula 1, as claimed in claim 1, in which
R1 is 1-4C-alkyl,
R2 is 1-4C-alkyl,
R3 is hydrogen,
R4 is hydrogen, 1-4C-alkyl, 1-4C-alkoxy-1-4C-alkyl, hydroxy-1-4C-alkyl or 1-4C-alkylcarbonyl,
R5 is hydrogen,
R6 is hydrogen,
or a salt thereof.

6. A compound of the formula 1 as claimed in claim 1, characterized by the formula 1a, 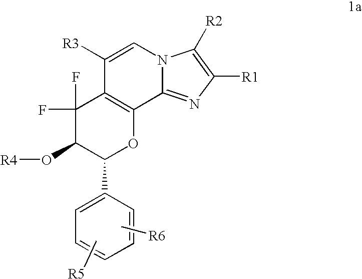
in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkinyl, fluoro-1-4C-alkyl, fluoro-1-4C-alkoxy-1-4C-alkyl or hydroxy-1-4C-alkyl,
R2 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 2-4C-alkenyl, 2-4C-alkinyl, fluoro-1-4C-alkyl, fluoro-1-4C-alkoxy-1-4C-alkyl or hydroxy-1-4C-alkyl,
R3 is hydrogen, halogen, 1-4C-alkyl, fluoro-1-4C-alkyl, 3-7C-cycloalkyl, 3-7C-cycloalkyl-1-4C-alkyl, 1-4C-alkoxy-1-4C-alkyl, fluoro-1-4C-alkoxy-1-4C-alkyl or the group —CO—NR31R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,
R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,
R5 is hydrogen, halogen, 1-4C-alkyl or fluoro-1-4C-alkyl,
R6 is hydrogen, halogen, 1-4C-alkyl or fluoro-1-4C-alkyl,
or a salt thereof.

7. A compound of the formula 1a as claimed in claim 6, in which
R1 is hydrogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,
R2 is hydrogen, halogen, 1-4C-alkyl, 3-7C-cycloalkyl, 3-7C-cycloalkyl-1-4C-alkyl, 2-4C-alkenyl, 2-4C-alkinyl or fluoro-1-4C-alkyl,
R3 hydrogen, halogen, 1-4C-alkyl, fluoro-1-4C-alkyl, 3-7C-cycloalkyl, 1-4C-alkoxy-1-4C-alkyl, fluoro-1-4C-alkoxy-1-4C-alkyl or the group —CO—NR31R32, where
R31 is hydrogen, hydroxyl, 1-7C-alkyl, 3-7C-cycloalkyl, hydroxy-1-4C-alkyl or 1-4C-alkoxy-1-4C-alkyl and
R32 is hydrogen, 1-7C-alkyl, hydroxy-1-4C-alkyl, 3-7C-cycloalkyl or 1-4C-alkoxy-1-4C-alkyl, or where
R31 and R32 together, including the nitrogen atom to which both are bonded, are a pyrrolidino, hydroxy-pyrrolidino, azetidino, aziridino, piperidino, piperazino, N-1-4C-alkylpiperazino or morpholino group,
R4 is hydrogen, 1-4C-alkyl, 3-7C-cycloalkyl, 1-4C-alkoxy-1-4C-alkyl, fluoro-1-4C-alkyl, fluoro-1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,
R5 is hydrogen, halogen, 1-4C-alkyl or fluoro-1-4C-alkyl,
R6 is hydrogen, halogen, 1-4C-alkyl or fluoro-1-4C-alkyl,
or a salt thereof.

8. A compound of the formula 1a as claimed in claim 6, in which
R1 is 1-4C-alkyl,
R2 is hydrogen or 1-4C-alkyl,
R3 is hydrogen or 1-4C-alkoxy-1-4C-alkyl,
R4 is hydrogen, 1-4C-alkyl, 3-7C-cycloalkyl, 1-4C-alkoxy-1-4C-alkyl, fluoro-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,
R5 is hydrogen,
R6 is hydrogen,
or a salt thereof.

9. A compound of the formula 1a as claimed in claim 6, in which
R1 is 1-4C-alkyl,
R2 is hydrogen or 1-4C-alkyl,
R3 is hydrogen,
R4 is hydrogen, 1-4C-alkoxy-1-4C-alkyl, hydroxy-1-4C-alkyl, 1-4C-alkoxy-1-4C-alkylcarbonyl, mono- or di-1-4C-alkylamino-1-4C-alkylcarbonyl, 1-4C-alkylcarbonyl or 1-4C-alkoxycarbonyl,
R5 is hydrogen,
R6 is hydrogen,
or a salt thereof.

10. A compound of the formula 1a as claimed in claim 6, in which
R1 is 1-4C-alkyl,
R2 is 1-4C-alkyl,
R3 is hydrogen,
R4 is hydrogen, 1-4C-alkyl, 1-4C-alkoxy-1-4C-alkyl, hydroxy-1-4C-alkyl or 1-4C-alkylcarbonyl,
R5 is hydrogen,
R6 is hydrogen,
or a salt thereof.

11. The compound (8R,9R)-7,7-Difluoro-2,3-dimethyl-8-hydroxy-9-phenyl-7H-8,9-dihydro-pyrano(2,3-c]imidazo[1,2-a]pyridine or a salt thereof.

12. The compound (8R,9R)-7,7-Difluoro-2,3-dimethyl-8-(2-methoxy-acetyloxy)-9-phenyl-7H-8,9-dihydro-pyrano[2,3-c]imidazo[1,2-a]pyridine or a salt thereof.

13. The compound (8R,9R)-7,7-Difluoro-2,3-dimethyl-8-(methoxy-carbonyloxy)-9-phenyl-7H-8,9-dihydro-pyrano[2,3-c]imidazo[1,2-a]pyridine or a salt thereof and its salts.

14. A pharmaceutical composition comprising a compound as claimed in claim 1 and/or a pharmacologically acceptable salt thereof together with a pharmaceutically acceptable auxiliary and/or excipient.

15. (canceled)

16. A method of preventing or treating a gastrointestinal disorder in a patient comprising administering to a patient in need thereof a therapeutically effective amount of a compound as claimed in claim 1, or a pharmacologically acceptable salt thereof.
